(19) United States
(12) Patent Application Publication
(10) Pub. No.: US 2024/0118292 A1
(43) Pub. Date: Apr. 11, 2024

(54) Levels of CSF-Seeded alpha-Synuclein Oligomers reflect Parkinson's Disease Severity

(71) Applicant: Qatar Foundation for Education, Science and Community Development, Doha (QA)

(72) Inventor: Omar El-Agnaf, Doha (QA)

(21) Appl. No.: 18/378,967

(22) Filed: Oct. 11, 2023

Related U.S. Application Data
(60) Provisional application No. 63/415,144, filed on Oct. 11, 2022.

(51) Int. Cl. G01N 33/68

(52) U.S. Cl. CPC G01N 33/6896; G01N 2800/2814; G01N 2800/2835; G01N 2800/52

(57) ABSTRACT

The instant disclosure relates to methods for measuring disease progression in Parkinson's Disease and other neurodegenerative diseases.

### CROSS REFERENCE TO RELATED APPLICATIONS

This application claims the benefit of U.S. Provisional Application No. 63/415,144 filed Oct. 11, 2022, which is incorporated herein by reference in its entirety.

### BACKGROUND

The primary diagnostic criteria for Parkinson's disease (PD) rely mainly on a constellation of clinical symptoms, yet the neurodegenerative process is thought to begin many years before overt clinical symptoms are observed. Thus, there is a pressing need to identify at-risk patients prior to the onset of clinical features. In recent years, considerable efforts have been invested in identifying specific disease markers and developing diagnostic tools for early detection of synucleinopathies, many of which have focused on the detection of misfolded α-Synuclein (αSyn) aggregates in tissue and biological fluids. PD and dementia with Lewy bodies (DLB) are both associated with underlying Lewy body disease which represents the second most common neurodegenerative disorder after Alzheimer's disease (AD). Multiple System Atrophy (MSA) is another disease for which there is a lack of diagnostic tools. The neuropathological hallmark of Lewy body disease is the intracellular aggregation of the protein αSyn into spherical cytoplasmic inclusions (termed Lewy bodies or Lewy neurites).

Among the different assays established for detection of αSyn forms, enzyme linked immunosorbent assay (ELISA) is a simple and rapid technique that permits sensitive and specific quantification of the analytes of interest and is convenient for large scale screening in a clinical set-up. A possible limitation with ELISAs is that the analyte concentration may be so low that it falls below the limit of quantification for the ELISA.

Recent studies have demonstrated the ability of seed amplification assays (SAAs) to detect αSyn disease-associated aggregates in brain homogenates and CSF samples. However, SAAs, in their current format, are fluorescence based assays which are mainly binary tests (positive or negative), and do not provide any information regarding changes in levels of αSyn aggregates over the course of a disease or in response to treatment. Studies have shown that mere detection of αSyn in CSF is not a reliable marker of PD diagnosis or progression. A further limitation is that the αSyn test alone does not distinguish between Parkinson's and other disorders characterized by αsynuclein clumps, such as dementia with Lewy bodies (DLB) or multiple system atrophy (MSA). There is an unmet need for robust diagnostic biomarkers that can be used to monitor the course of PD, the course of MSA, or the course of DLB.

### SUMMARY

This disclosure provides assays combining αSyn SAA with oligomers-specific ELISA assays for obtaining quantitative information that reflects disease progression and/or severity of PD and/or DLB.

The present disclosure generally relates to methods of identifying disease-associated αSyn aggregates as biomarkers of PD clinical stages. In one aspect, provided herein is a method for monitoring Parkinson's Disease (PD) progression or Dementia with Lewy Bodies (DLB) progression comprising
(i) detecting αSyn aggregation in a sample comprising αSyn oligomers using a seed amplification assay (SAA); and
(ii) measuring the amount of αSyn in the sample of step (i) with an enzyme linked immunosorbent assay (ELISA) at a time point when the amount of αSyn oligomers in the sample of step (i) exceeds the ELISA limit of quantification.

Further aspects comprise methods of treating identified diseases or conditions based on the readout from the multiplexed assays described herein.

### BRIEF DESCRIPTION OF THE FIGURES

FIG. 1. Flowchart of the study cohorts. A flowchart presenting the number of cases per cohort, and the primary question answered by each samples' set is shown.

FIG. 2a. SAA and ELISA analysis of the pilot BH samples set. RT-QuIC reactions seeded in triplicate with brain homogenate (BH) from control (Ctrl), Parkinson's disease (PD) or dementia with Lewy bodies (DLB) cases. The solid line of each sample trace represents the average thioflavin T (ThT) signal of triplicate wells. The shaded area represents the standard error (±SD). FIG. 2b. Comparison of Imax from each group. FIG. 2c. Comparison of area under the ThT curve (ThT AUC) for each group FIG. 2d. Comparison of seeded αSyn oligomers (FIG. 2d) for each group. The dots represent the single cases, and the lines reflect the group's average. Statistical analysis was not conducted at this stage due to the small sample size, however, the differences between the groups are clearly pronounced.

FIG. 3a. SAA and ELISA analysis of the validation BH samples set. RT-QuIC reactions seeded in triplicate with brain homogenate (BH) from subjects with αSyn pathology (PD, n=1 and DLB, n=4), or without αSyn pathology (AD, n=3 and Ctrl, n=2). The solid line represents the average ThT signal per group. FIG. 3b. Monitoring the changes of αSyn oligomers' levels over multiple time points (0, 48, 72, 96, and 120 hr) for each group for ELISA. FIG. 3c. Monitoring the changes of αSyn oligomers' levels over multiple time points (0, 48, 72, 96, and 120 hr) for each group for SAA.

FIG. 4a. RT-QuIC reactions seeded in triplicate with CSF from control and PD subjects. RT-QuIC reactions seeded in triplicate with CSF samples from Ctrl subjects and patients with PD. The solid line represents the average ThT signal of triplicate wells. The shaded area represents the standard error. FIG. 4b. Comparison of the mean maximum fluorescence at SAA endpoint. FIG. 4c. Comparison of the RFU at 20 h of the assay run for each diagnostic group. FIG. 4d. Comparison of seeded CSF αSyn oligomers at 20 h of the assay run for each diagnostic group. FIG. 4e. Comparison of total CSF αSyn oligomers at 20 h of the assay run for each diagnostic group. FIG. 4f. Floating bars show the min to max with the line at the mean. ROC curves based on logistic regression analyses for the classification of PD patients versus Ctrls based on various predictors; seeded CSF o-αSyn oligomers, the mean maximum fluorescence at 60 h (Imax RFU), the mean maximum fluorescence at 20 h, and area under the ThT curve (ThT AUC). FIG. 4g. Plot for calculated values of ROC area under the curve (ROC AUC). FIG. 4h. Plot for sensitivity. FIG. 4i. Plot for specificity.

FIG. 5a. Correlation analysis in discovery and validation cohorts. Scatter plots showing the correlation analysis in the discovery cohort between CSF seeded αSyn oligomers and UPDRS-motor scores. FIG. 5b. Scatter plots showing the correlation analysis in the discovery cohort between CSF seeded αSyn oligomers and H&Y scores, respectively. FIG. 5c. Scatter plots showing the correlation analysis in the validation cohort between UPDRS-motor with seeded CSF αSyn oligomers. FIG. 5d. Scatter plots showing the correlation analysis in the validation cohort between H&Y scores with seeded CSF αSyn oligomers. The subplots FIG. 5A-1, FIG. 5B-1, FIG. 5C-1, and FIG. 5D-1 present the same dataset excluding extreme data points highlighted with the circle. The solid line highlights the calculated regression line. P value and Spearman rank correlation (r) is displayed for each correlation.

### DETAILED DESCRIPTION

PD diagnosis is not always accurate and potentially overlaps with other neurodegenerative disorders. A previous study (Shahnawaz M. et al., JAMA Neural 2017; 74:163-172) reported PMCA (a technique analogous to αSyn SAA) at time T50 to correlate with the disease severity in PD patients assessed by H&Y scale (r=−0.54, P=0.006) However, in our hands, SAA kinetic parameters failed to correlate with PD-specific clinical features in two CSF cohorts that were included in our study. A recent comparative study (Russo M. J. et al., Acta Neuropathol Commun 2021; 9:179), utilizing the Parkinson's Progression Markers Initiative (PPMI) longitudinal cohort, also accentuated the shortcomings of αSyn SAA alone at reflecting disease severity, clinical features, or clinical subtypes.

Accordingly, described herein are assays that address certain shortcomings of current methods. Initially, misfolded αSyn in pathologically well-characterized human brain tissues were measured using SAA. Further analyses of SAA end-products using Syn-02 ELISA revealed a correlation between the two assays, as both PD and DLB groups scored positive in SAA, and also showed high levels of seeded αSyn oligomers in the ELISA. Likewise, controls scored negative on SAA and no seeded αSyn oligomers were detected above the ELISA limit of quantification.

The analyses were expanded to a larger set of human brain tissues, and the methods described herein showed consistency in distinguishing PD and DLB patients from Ctrl or AD subjects. Moreover, the amplification of misfolded αSyn in from PD and DLB was robustly detected by ELISA at an earlier time point compared to SAA alone. For instance, detection of misfolded αSyn by ELISA was observed between about 50 to about 90 hours, or between about 70 to about 90 hours, of seed amplification, whereas a similar segregation was only achieved at 120 hours using SAA alone. Accordingly, the methods described herein advantageously provide an early and quantitative discrimination between the control vs diseased groups and/or between different neurodegenerative conditions. The SAA and ELISA analyses can be conducted sequentially, in any order, or simultaneously in parallel (multiplexed).

### Definitions

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value. In some embodiments “about” indicates that the referenced measurement may be ±10% of the recited value. In some embodiments “about” indicates that the referenced measurement may be ±5% of the recited value. In some embodiments “about” indicates that the referenced measurement may be ±1% of the recited value.

“αSyn” means α-Synuclein protein which is a predominantly neuronal protein of approximately 14 KDa size. αSyn may be wild type, or a recombinant protein.

“αSyn oligomers” refers to oligomers that comprise several to dozens of monomers of Syn. αSyn oligomers are highly heterogeneous with respect to molecular weight and structure.

“αSyn aggregation” or “αSyn aggregate” occurs when a plurality of αSyn oligomers interact with each other. αSyn oligomers may be “on pathway” oligomers or “off pathway” oligomers and both pathways lead to “αSyn aggregates”. In some embodiments, αSyn aggregates are formed via “on pathway” αSyn oligomers, which typically form protofibrils or fibrils. In some embodiments, αSyn aggregates are formed via “off pathway” oligomers which typically form varied shapes/structures. Non-limiting examples of αSyn oligomers or αSyn aggregates that may be detected and/or quantified by the methods described herein include αSyn oligomers or αSyn aggregates described in El-Agnaf O. M. et al., FASEB J 2006; 20:419-425, Vaikath N. N. et al. Neurobiol Dis 2015; 79:81-99, Majbour N. K. et al., Mov Disord 2016; 31:1535-1542.

“Seed amplified assays” or “SAA” refers to a technique for detecting αSyn aggregates when the analyte levels are low in samples. In some embodiments, in certain SAAs, the initial very low number of fibrils act as seeds for the amplification of the fibril mass to a detectable level. Amplification is achieved by adding nonaggregated monomeric αSyn as reactant to the sample of interest. The seeds in the sample will recruit αSyn monomers and elongate into longer fibrils. Shaking the sample induces fragmentation of the elongated fibrils, which increases the number of fibrils. These newly created fibrils again grow by recruiting monomers, and this cycle continues. This combination of events leads to an increase in the number of fibrils and thus the fibril mass. In the SAA, the amplification is monitored using the fluorescent amyloid binding dye thioflavin T (ThT). Upon binding to the fibril, ThT becomes strongly fluorescent, which allows for the use of the total fluorescence intensity as a readout for the presence of fibrils once the seeds are sufficiently amplified.

“Progression” of a disease refers to the change in a subject's disease state over the course of the disease. Disease progression may be determined by a physician who may administer certain diagnostic tests (e.g., Hoehn and Yahr (H&Y) scale, Unified Parkinson's Disease Rating Scale (UPDRS), or other suitable tests).

A “relative fluorescence unit” or RFU is a unit of measurement used in analysis which employs fluorescence detection. There are no industry-wide rules for establishing minimum RFU threshold values. Each laboratory, in general, establishes its own threshold levels as part of its validation procedure. In one embodiment, an RFU is as described in Table 2.

“RT-QuIC” refers to real-time quaking-induced conversion assays. This technique exploits the ability of the misfolded pathological form of αSyn protein found, for example, in cerebrospinal fluid (CSF) to induce conversion of normal αSyn to the misfolded form, which subsequently aggregates. The formation of these aggregates is monitored in real time using fluorescent dyes.

“Non-human” species” refers to pre-clinical species generally known in the art for testing pharmaceutical agents that are under development. Non-human species include, and are not limited to, rats, mice, dogs, primates, cats, rabbits, and any other species known to one of skill in the art.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference made to a patent document or other publication in this specification serves as an incorporation herein by reference of the entire content of such document or publication.

Embodiments of this disclosure are illustrative. Accordingly, the present disclosure is not limited to that precisely as shown and described.

### Methods

This disclosure provides a method for monitoring Parkinson's Disease (PD) progression or Dementia with Lewy Bodies (DLB) progression. The method comprises two assays that may be run sequentially, in any order, or, simultaneously in parallel. In a first assay, the method comprises detecting αSyn aggregation in a sample comprising αSyn oligomers using a seed amplification assay (SAA). A second assay comprises measuring the amount of αSyn in the seed amplified sample with an enzyme linked immunosorbent assay (ELISA) at a time point when the amount of αSyn oligomers in the sample exceeds the ELISA limit of quantification. The ELISA read out can be used to quantify the amount of αSyn in the sample. The time point when the αSyn oligomers in the sample can be detected by the ELISA may vary depending on factors such as dilution of the sample, the amount of analyte in the CSF. Preferably, the ELISA assay is run at the earliest time point when the amount of αSyn oligomers in the sample exceeds the ELISA limit of quantification.

In some embodiments, the sample is a cerebro spinal fluid (CSF) sample, a brain homogenate (BH) sample, a whole blood sample, a plasma sample, a serum sample, a saliva sample, or a urine sample.

In some embodiments, the sample is a cerebro spinal fluid (CSF) sample, which is seeded, and the progression of PD is monitored. In other words, the sample is a CSF sample. This CSF sample is seeded into a reaction mixture containing components for amplification. When the amplification has run for a sufficient length of time, αSyn oligomers in the reaction mixture are detectable using ELISA, and thus the progression of PD is monitored. In some embodiments, the ELISA assay is conducted at a time point between about 5 hours to about 120 hours after the seeding of the sample. In some embodiments, the ELISA assay is conducted at a time point between about 10 hours to about 90 hours after the seeding of the sample. In some embodiments, the ELISA assay is conducted at a time point between about 15 hours to about 60 hours after the seeding of the sample. In some embodiments, the ELISA assay is conducted at a time point between about 15 hours to about 45 hours after the seeding of the sample. In some embodiments, the ELISA assay is conducted at a time point between about 15 hours to about 25 hours after the seeding of the sample. In some embodiments, the ELISA assay is conducted ata time point about 20 hours after the seeding of the sample.

In some embodiments, the sample is a brain homogenate (BH) sample, which is seeded, and the progression of PD or DLB is monitored. In other words, the sample is a BH sample. This BH sample is seeded into a reaction mixture containing components for amplification. When the amplification has run for a sufficient length of time, αSyn oligomers in the reaction mixture are detectable using ELISA, and thus the progression of PD or DLB is monitored. In some embodiments, the ELISA assay is conducted at a time point between about 5 hours to about 120 hours after the seeding of the sample. In some embodiments, the ELISA assay is conducted at a time point between about 10 hours to about 90 hours after the seeding of the sample. In some embodiments, the ELISA assay is conducted at a time point between about 25 hours to about 75 hours after the seeding of the sample. In some embodiments, the ELISA assay is conducted at a time point between about 55 hours to about 65 hours after the seeding of the sample. In some embodiments, the ELISA assay is conducted at a time point about 60 hours after the seeding of the sample.

In some embodiments, the said ELISA comprises antibodies that recognize αSyn oligomers or αSyn aggregates, and do not recognize αSyn monomers. In some of such embodiments, the ELISA assay may comprise antibodies which recognize certain conformations within the αSyn aggregates, or antibodies which recognize αSyn oligomers.

In some embodiments, the levels of CSF seeded αSyn oligomers measured by the methods described herein correlate with the severity of the clinical symptoms of PD as measured by the Hoehn and Yahr (H&Y) scale and the Unified Parkinson's Disease Rating Scale (UPDRS) motor scores.

In some embodiments, the levels of BH seeded αSyn oligomers measured by the methods described herein correlate with the severity of the clinical symptoms of PD or DLB as measured by diagnostic testing.

Provided is an assay for measuring the effect of test molecules on αSyn aggregation, the assay comprising
(i) obtaining a sample from a subject;
(ii) detecting αSyn aggregation in the sample using a seed amplification assay (SAA); and
(iii) measuring the amount of αSyn in the sample of step (ii) with an enzyme linked immunosorbent assay (ELISA) at a time point when the amount of αSyn oligomers in the sample of step (ii) exceeds the ELISA limit of quantification.

In some embodiments, the assay is a high throughput assay for rapid testing of molecules in a research program. “Test molecules” as used herein refers to molecules that are in pre-clinical or clinical testing/research and development, and in some embodiments, may be potential candidates for regulatory approval. In some embodiments, “test molecules” are small molecules. In some embodiments, “test molecules” are antibodies. In some embodiments, “test molecules” are mRNA. In some embodiments, “test molecules” are antisense RNA including small interfering or short or silencing RNA (siRNA).

In some embodiments, the sample in the assay is a cerebro spinal fluid (CSF) sample, a brain homogenate (BH) sample, a whole blood sample, a plasma sample, a serum sample, a saliva sample, or a urine sample.

In some embodiments of the assay, the test molecules affect αSyn aggregation directly or indirectly. As used herein, “directly” affecting αSyn aggregation refers to test molecules that may interfere with formation of αSyn oligomers or aggregates. As used herein, “indirectly” affecting αSyn aggregation refers to test molecules that may interfere with processes upstream or downstream of αSyn aggregation.

In some embodiments, the test molecules that affect αSyn aggregation directly or indirectly are selected from antibodies, small molecules that interfere with αSyn aggregation, or molecules that affect pathways associated with GBA gene mutations or LRRK2 gene mutations.

In some embodiments, the assay is used in pre-clinical testing. In some embodiments of pre-clinical assay testing, the sample is a BH sample obtained from a non-human species.

In some embodiments of pre-clinical assay testing, the sample is a CSF sample, a whole blood sample, a plasma sample, a serum sample, a saliva sample, or a urine sample obtained from a non-human species.

In some embodiments, the assay is used in human clinical trials. In some embodiments of clinical assay testing, the sample is a cerebro spinal fluid (CSF) sample, a whole blood sample, a plasma sample, a serum sample, a saliva sample, or a urine sample obtained from a human subject.

Also provided herein is a kit for quantification of αSyn aggregates comprising
(i) a container comprising untagged or tagged monomeric full length αSyn, C-terminally truncated αSyn, N-terminally truncated αSyn, or fragments of αSyn; and
(ii) an ELISA assay that recognizes αSyn oligomers or αSyn aggregates and does not recognize αSyn monomers.

Also provided herein is a method for treating a patient suffering from PD or DLB or MSA, the method comprising (i) identifying the PD or DLB or MSA progression according to the methods described herein; and (ii) adjusting the patient's therapeutic regimen based on the identified PD or DLB or MSA progression.

### Examples

This study combines a seed amplification assay (SAA) and an enzyme-linked immunosorbent assay (ELISA) in order to provide a quantitative test readout that reflects the clinical profiles of PD patients. Two sets of human brain homogenates (pilot and validation sets) are initially explored, and then verified with two independent human CSF cohorts: a discovery cohort (62 PD, and 34 control) and a validation cohort (49 PD and 48 control).

### Study Design

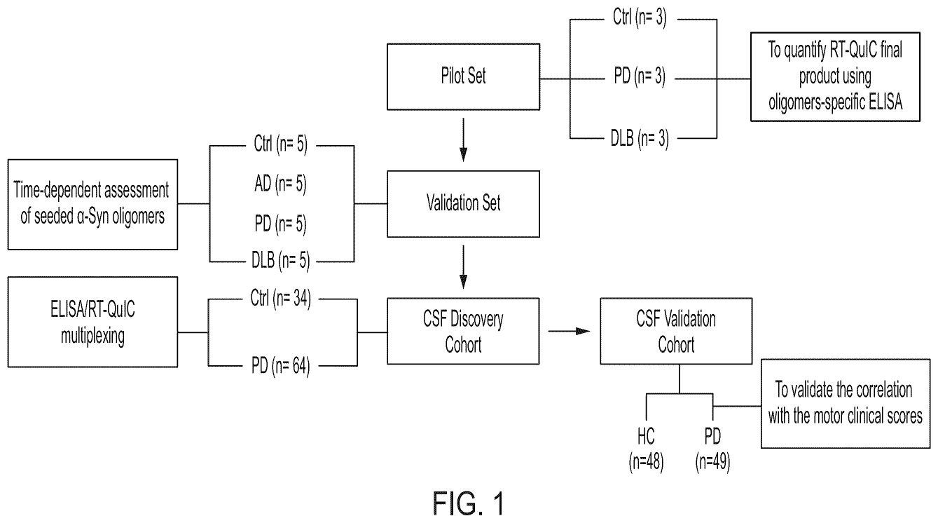

First, we use a pilot set of post-mortem human brain homogenates (BH), followed by a validation set of similar tissues. Next, we explore our approach in a discovery human CSF cohort of PD patients and Control (Ctrl) subjects. We then validate the correlation with clinical scores noted in the discovery cohort in a second independent human CSF cohort termed “validation cohort”. The study flow is shown in FIG. 1.

### Brain Homogenate Preparation

Frozen post-mortem samples of the frontal cerebral cortex (Brodmann area 9) from clinically diagnosed and neuropathologically-confirmed cases are obtained from Newcastle Brain Tissue Resource, Translational and Clinical Research Institute, Newcastle University, UK and processed as previously described by Vaikath N. N. et al., Neuropathol Appl Neurobiol 2019; 45:597-608. The test cases are divided into two sets, a pilot set (Ctrl=3, PD=3, and DLB=3), and a validation set (Ctrl=5, AD=5, PD=5, and DLB=5). Briefly, tissue samples are homogenized with a glass tissue homogenizer at 10% (w/v) on ice in tris-buffered saline (TBS) (20 mM Tris-HCl pH 7.4, 150 mM NaCl) containing a cocktail of protease and phosphatase inhibitors and 5 mM ethylenediaminetetraacetic acid (EDTA) (Thermo Fisher Scientific). Samples are then centrifuged at 3000×g, at 4° C. for 30 min. The supernatant is collected, and total protein concentration is measured for each sample using bicinchoninic acid (BCA) protein assay kit (Pierce, Thermo Scientific). All brain homogenates (BH) are aliquoted and stored at −80° C. till further use.

### Study Participants and CSF Sampling

Discovery Cohort

Patient selection criteria and the method of CSF collection are as described in previous publications (Aasly J. O. et al., Ann Neural 2005; 57:762-765; Majbour N. K. et al., Transl Neurodegener 2020; 9:15; Aasly J. O., Front Aging Neurosci 2014; 6:248). In total, 64 patients with idiopathic PD and 34 age-matched controls are included in the current study. As this cohort is part of a larger cohort recruited at St. Olav's Hospital at the University Hospital of Trondheim in Norway studying LRRK2 mutations, the control group is composed of first-degree relatives of LRRK2 mutation carriers for who are not carrying LRRK2 mutations. PD clinical diagnoses are made by experienced senior clinicians based on known guidelines and disease stage is assessed according to the Hoehn and Yahr (H&Y) scale. Lumbar puncture is performed in the morning between 8 and 10 am following an overnight fast. CSF is collected and aliquoted in 1.2-1.5 mL low-binding tubes, and one vial is sent for routine laboratory analysis (i.e., white and red blood cell count, total protein and glucose levels, according to the Parkinson's Progression Markers Initiative [PPMI] protocol). The majority of the vials are frozen fewer than 15 min after collection following centrifugation at 2000 g at 4° C. then sub-aliquoted and stored at −80° C. until further analysis. All patients provide written informed consent, and the study is approved by the Regional Committee for Medical and Health Research Ethics (Ethical committee of Central Norway number 34272). Frozen CSF 0.5-1 ml aliquots are shipped on dry ice. Samples are kept frozen before the shipping and also during the entire period of transportation until analysis.

Validation Cohort

A detailed description of the cohort has been published elsewhere (van Dijk K. D. et al., Eur J Neural 2014; 21:388-394). The cohort includes 49 patients with PD that attended the outpatient clinic for movement disorders at the VU University Medical Center (VUMC) between September 2008 and February 2011, and 48 age-matched healthy controls (HC) (recruited via an advertisement in the periodical of the Dutch Foundation). All PD patients included are able to understand the study aim and procedures, and fulfill the United Kingdom Parkinson's Disease Society Brain Bank clinical diagnostic criteria. Mini-Mental State Examination and/or neuropsychological assessment in the patients did not indicate dementia. Severity of symptoms and disease stage in the ‘on’ state is rated using the Unified Parkinson's Disease Rating Scale-Part-III (UPDRS-III) and the modified Hoehn and Yahr (H&Y) classification.

### Recombinant αSyn Protein Expression and Purification

Recombinant human full-length αSyn protein (1-140) is expressed from the pRKI72 plasmid containing full length cDNA for the human SNCA gene as previously described (Vaikath N. N., Neuropathol Appl Neurobiol 2019; 45:597-608). Briefly, recombinant αSyn protein is expressed untagged in E. Coli BL21 (DE3) and purified using size exclusion (Hiload 16/600 Superdex 200 μg) and Mono Q anion exchange chromatography. Recombinant αSyn is then dialyzed against phosphate buffer saline (PBS, pH 7.4), filtered through (100-kDa spin filter) to remove any preformed aggregates, and the protein concentration is estimated using BCA protein assay kit (Pierce, Thermo Scientific). The protein is then aliquoted and stored frozen until use. The protein aliquots are not subjected to any thaw-freeze cycle prior to use in SAA.

### αSyn Seed Amplification Assay

For brain homogenate (BH) samples, 160 μL of a reaction mixture composed of 0.1 M piperazine-N, N′ bis (ethanesulfonic acid) (PIPES), pH 6.5, 0.5 M sodium chloride (NaCl), 10 μM Thioflavin T (ThT), and 0.1 mg/mL wild-type untagged monomeric αSyn (filtered through a 100 kD MWCO filter immediately prior to use), are distributed in 96-well black plates with clear bottom (Nunc, Thermo Fisher) at a final volume of 200 μL per well. For each test, we loaded 40 μl of BH of 0.1 mg/mL total protein concentration. The plate is then sealed with a sealing tape and incubated in Omega FIUOstar plate reader (BMG Labtech, Aylesbury, Buckinghamshire, UK) at 37° C. for 120 hours with intermittent shaking cycles: double orbital with 1 min shake (500 rpm,) and 15 min rest throughout the indicated incubation time.

For CSF seeding assay, wells are pre-loaded with α silica beads (Sigma-Aldrich), and 85 μL of a reaction mix prepared to give final reaction concentrations of 40 mM phosphate buffer (pH 8.0), 170 mM NaCl, 0.1 mg/mL recombinant monomeric αSyn (filtered through a 100 kD MWCO filter immediately prior to use), 10 μM ThT and 0.0015% sodium dodecyl sulfate (SDS) were distributed according to the plate layout. Then 15 μL CSF per sample is spiked in triplicates into corresponding wells. The plate is then sealed with a sealing tape and incubated in Omega FLUOstar plate reader (BMG labtech, Aylesbury, Buckinghamshire, UK) at 42° C. with intermittent shaking cycles: double orbital with 1 min shake (500 rpm,) and 1 min rest throughout the indicated incubation time. For both protocols, ThT fluorescence readings are taken every 45 min with a bottom read using 450±10 nm (excitation) and 480±10 nm (emission) wave-lengths. The sample is considered positive if 2 or more of the replicates are above the calculated threshold. The threshold is calculated as the average fluorescence for all samples within the first 10 h of incubation, plus 3 times the Standard Deviations (SD).

For BH SAA, the optimal time point is identified by collecting aliquots of 5 μL from each well for each sample at 0, 48, 72, 96, and 120 h of the assay total run time (120 h). For CSF SAA, the same volume is aliquoted at 0, 20 and 60 h of the assay total run time (60 h). All aliquots are stored at −80° C. until analysis in ELISA.

### αSyn Oligomer-Specific ELISA

All samples are analyzed using an oligomer-specific ELISA (described in Majbour N. K. et al., Mov Disord 2016; 31:1535-1542; Vaikath N. N., Neurobiol Dis 2015; 79:81-99) with minor modifications. Syn-02 is used for capture (at 0.5 μg/mL, overnight incubation at 4° C.), and biotinylated Syn-02 (at 0.5 μg/mL, 1 h incubation at 37° C.) is used for detection with HRP-conjugated streptavidin (Sigma Aldrich) (at 1:5000 dilution, 30 min incubation at 37° C.) as the reporter. Plates are initially blocked to eliminate non-specific signal for 1 h at 37° C., and samples are diluted at 1:1000 in 50% radioimmunoprecipitation assay (RIPA) buffer prior to loading, and incubated for 1 h at 37° C. Chemiluminescence expressed in relative light units is immediately measured using a PerkinElmer Envision plate reader (PerkinElmer, Finland). Specified calibrators are used to generate an 8-point standard curve to which a 4-parameter logistic (4PL) curve of all plates is fitted and used to quantify unknown concentrations using GraphPad Prism software. The concentrations of oligomeric and total αSyn are extrapolated from corresponding standard curves. For each case analyzed by SAA, individual replicas are tested in duplicates using ELISA (i.e. 3 replicas×2 duplicates per case) and the average is calculated. Each ELISA run is performed using a 384-well maxisorp plate and completed within ˜4-5 h.

### Statistical Analyses

GraphPad Prism (version 8.3.0) software is used to analyze and plot both SAA calculated kinetic parameters (maximum intensity of fluorescence (Imax) at final point (60 h), and at 20 h of the assay run, time needed to reach 50% of the maximum aggregation (T50), and area under the ThT fluorescence sigmoid curve (ThT AUC)) for CSF samples, and ELISA quantified αSyn levels (total and seeded αSyn oligomers). IBM SPSS software (version 24.0, Chicago, IL, USA) is used for receiver operating curve (ROC) analyses. All calculated parameters are tested for normality and deemed inappropriate for parametric analyses. Therefore, the Mann-Whitney U-test is used for comparisons between PD and HC diagnostic groups for the named variables. Spearman correlations are performed to explore possible associations between seeded αSyn levels, SAA kinetic parameters and PD clinical stage assessed. P<0.05 is set as the level of statistical significance. The sensitivity and specificity scores for ELISA and SAA are also calculated for the study cohorts.

### Study Observations:

(A) the Study Shows that ELISA Confers High Sensitivity and Specificity for SAA End Product Seeded by Brain Homogenates from Patients with PD and DLB

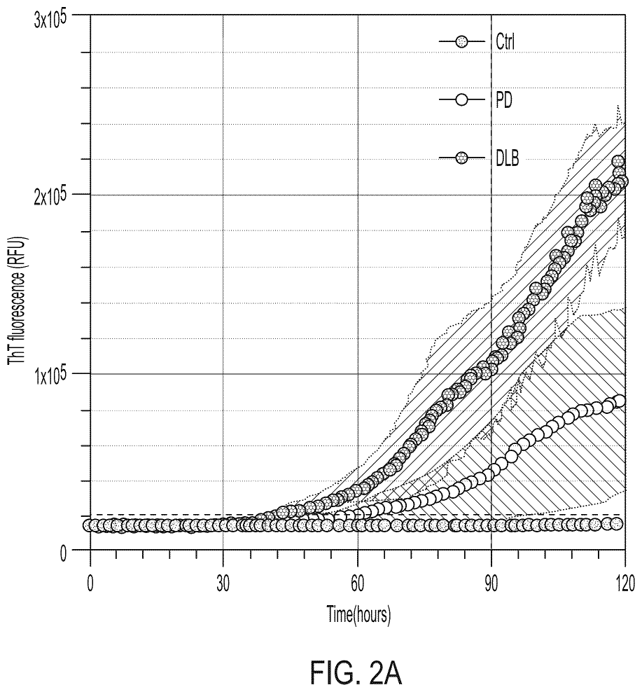

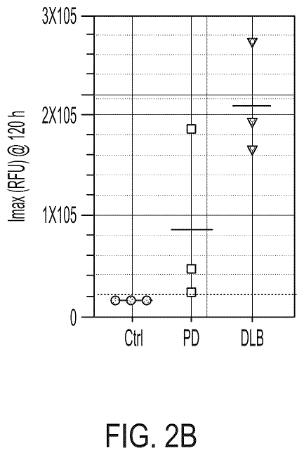

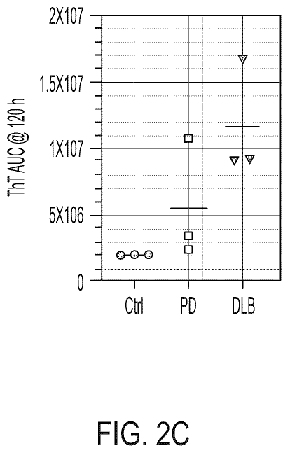

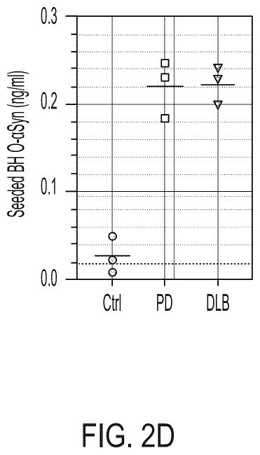

The in vitro assembly of recombinant monomeric wild type human αSyn is initially seeded with BH from frontal cortex in a pilot set of samples comprising three cases per group of PD, DLB and Control (Ctrl) subjects. A lag phase of about 40-60 h is observed (FIG. 2a), followed by a rapid increase in fluorescence and a plateau at about 90-120 hours. DLB samples are observed to seed faster and result in higher fluorescence intensities than PD, whereas no increase in fluorescence is observed in the Ctrl group. Both Imax, and ThT AUC, of the SAA aggregation curve is calculated and the values are compared for the three groups. The DLB group shows a trend towards a higher Imax and AUC compared to the PD group, and both are higher compared to the Ctrl group with one PD sample overlapping with Ctrl (FIG. 2b, c). SAA quantified end product using ELISA, shows that the levels of seeded αSyn oligomers in both PD and DLB groups are higher than the Ctrl group with no overlap (FIG. 2d).

(B) Detection of Seeding Activity in Brain Homogenates of PD and DLB Using Oligomeric-ELISA is Achieved at Earlier Time Points

For SAA-ELISA multiplex protocol, the dilution factor is optimized (1:1000). The dilution buffer (50% RI PA) and time point cut-off (data not shown) are optimized. The cut-off is defined as the most appropriate time point time where positive samples can be robustly measured above the ELISA limit of quantification (LLoQ). On average, PD and DLB BH samples give a positive response within ˜55-65 h, and ˜15-25 h for PD CSF samples, thus 60 h and 20 h are selected as the assay optimal time point for BH and CSF, respectively. Time-dependent analyses are performed to validate our results from the pilot set and to detect seeded αSyn oligomers at the earliest possible time point. The cohort includes human brain tissue from Ctrl (n=5), AD (n=5), PD (n=5), and DLB (n=5) cases. αSyn SAA assay is carried out as described above, and the seeding activity of the brain homogenates is monitored by assessing the formation of early soluble αSyn oligomers, or αSyn aggregates, using oligomeric-ELISA (Majbour N. K. et al., Mol Neurodegener 2016; 11:7; Majbour N. K. et al., Mov Disord 2016; 31:1535-1542).

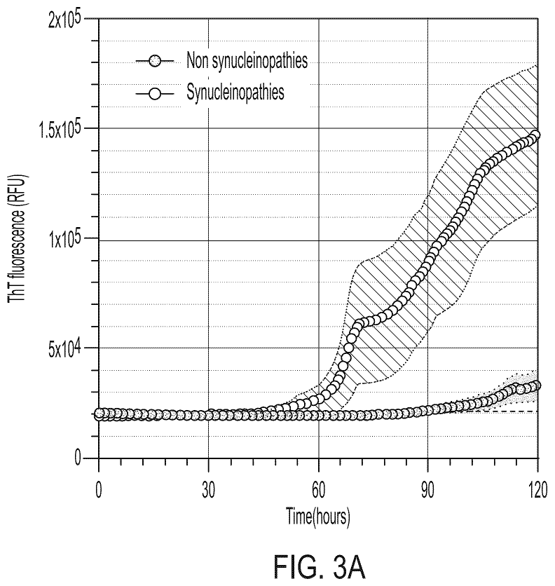

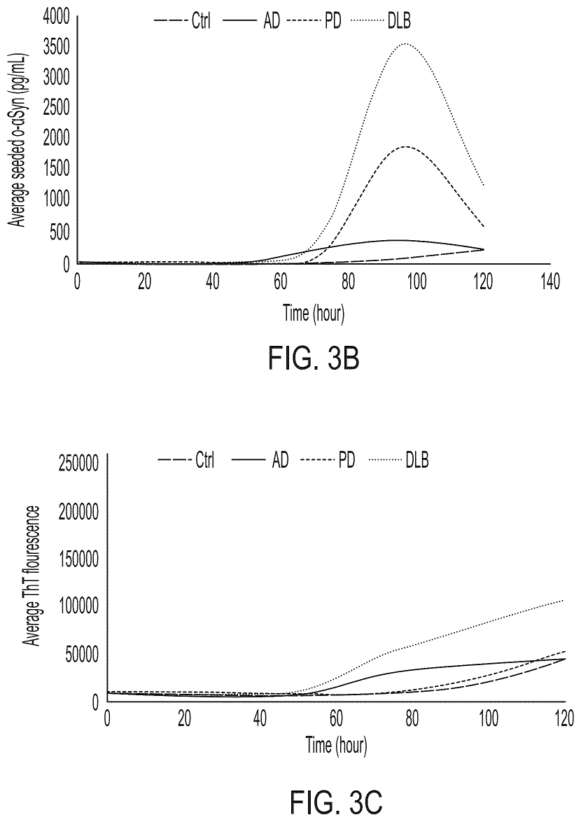

Samples are collected from individual replicas of each BH case at different time points over 120 h (0, 48, 72, 96, and 120 h), then frozen till ELISA analysis. As shown in FIG. 3a-c, levels of αSyn oligomers increased in a time-dependent manner up to a plateau at around 90 h. BH seeding activity is detected earlier with ELISA than SAA alone (72 versus 120 h), while maintaining optimal specificity for discriminating synucleinopathies from controls.

(C) CSF Seeded αSyn Oligomers Robustly Discriminates PD Patients from Controls

CSF samples from PD patients (n=62) and age-matched control subjects (Ctrl) (n=34) are analyzed to better understand the SAA-ELISA multiplex approach. The demographics of all subjects are shown in Table 1. As shown in Table 1, there is no significant difference in age or gender ratio between the 2 groups.

TABLE 1
Demographics and CSF biomarkers by diagnostic group

| | Discovery cohort | | Validation cohort | |
|---|---|---|---|---|
| | Ctrl | PD | Ctrl | PD |
| | (n = 34) | (n = 62) | (n = 48) | (n = 49) |
| Age (y), mean ± SD | 50 ± 16 | 57 ± 10 | 63.1 ± 10.3 | 63.3 ± 10.2 |
| Gender (male), n (%) | 16 (47%) | 33 (53%) | 16 (33%) | 30 (61%) |
| Disease duration (y), | NA | 4.5 ± 3.6 | NA | 5.8 ± 5.6 |
| mean ± SD | | | | |
| MoCA score | NA | 25.8 ± 2.9 | NA | NA |
| MMSE | NA | NA | 29.2 ± 1.1 | 28 ± 1.6 |
| H&Y score | NA | 2 ± 0.5 | NA | 2 ± 0.5 |
| UPDRS-III score | NA | 23.5 ± 9.1 | NA | 21.7 ± 8.6 |
| SAA t-αSyn (ng/mL) | 0.55 ± 0.20 | 0.80 ± 0.22 | | |
| SAA o-αSyn (ng/mL) | 0.05 ± 0.04 | 0.32 ± 0.18 | 0.016 ± 0.017 | 0.64 ± 0.60 |

Ctrl: healthy controls; H&Y: Hoehn and Yahr stage; MMSE: Mini-Mental State Exam; MoCA: Montreal Cognitive Assessment; NA: not applicable; o-αSyn: oligomeric α-synuclein; PD: Parkinson's disease; SAA: seed amplification assay; t-αSyn: total α-synuclein; UPDRS-III: Unified Parkinson's Disease Rating Scale.

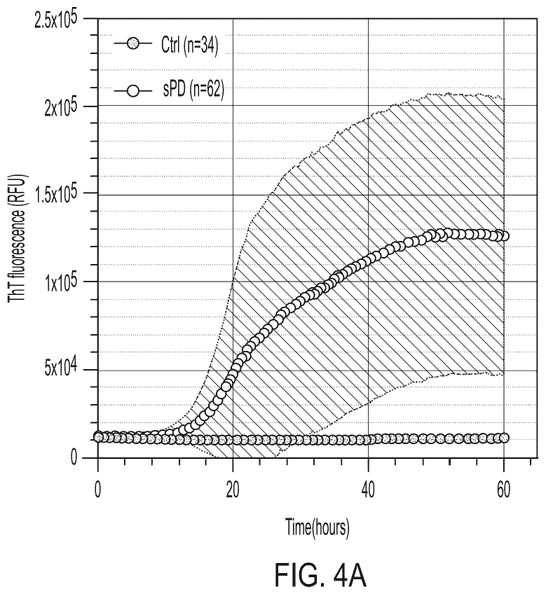

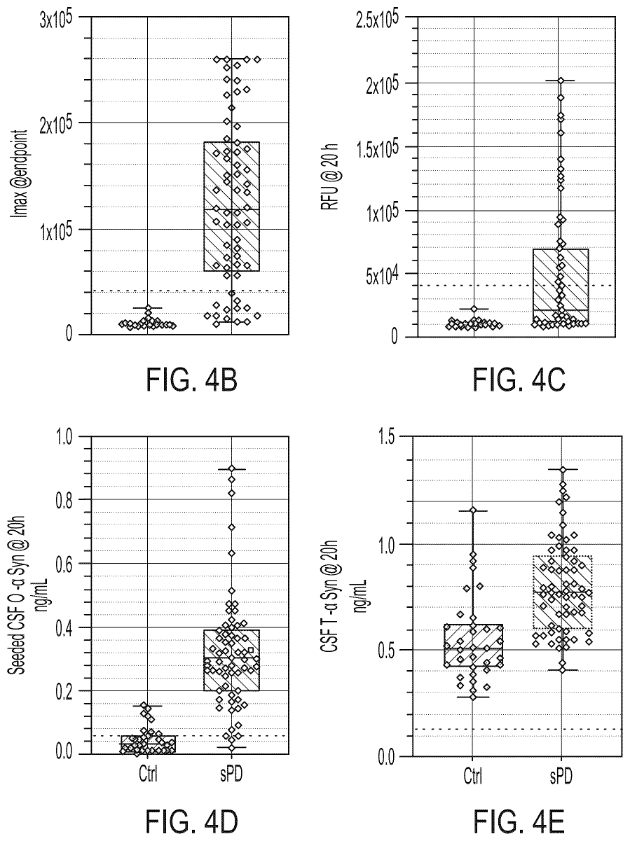

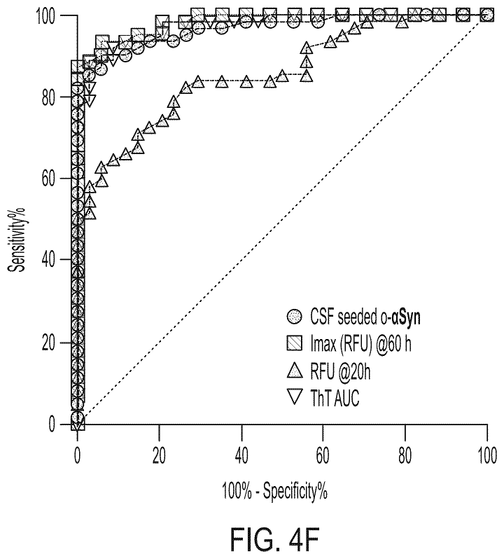

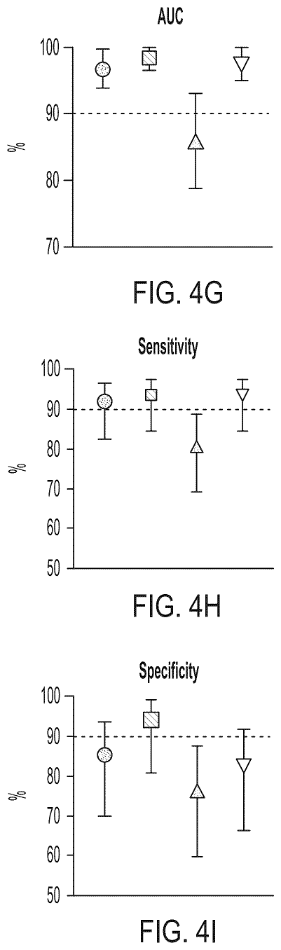

The time needed to reach 50% of the maximum aggregation (T50) varies greatly among PD positive CSF samples (FIG. 4a), whereas all Ctrl CSF samples with negative results based on the maximum fluorescence fail to reach the T50 value (FIG. 4a). In order to quantify the levels of αSyn oligomers in SAA seeded by CSF, samples from each replica of each case at 20 h of the assay run are analysed using ELISA assays for measuring total or αSyn oligomers. In comparing relative fluorescence units (RFU) values for SAA data, a notable overlap is noted between diagnostic groups at 20 h (FIG. 4c), which is significantly reduced at the end point (FIG. 4b). As a comparison, the levels of seeded αSyn oligomers are significantly higher in PD (mean±SD=321±180, n=62) compared to Ctrls (mean±SD=47±40, n=34) at 20 h (p<0.001, Mann-Whitney U test) (FIG. 4d), with minimal overlap compared to relative fluorescence units RFU at the same time point. The levels of total αSyn do not differ significantly between the two groups (mean±SD=799±219, n=62, and 554±201, n=3 for PD and Ctrl respectively) (FIG. 4e).

ROC analysis is performed to evaluate the accuracy of the diagnostic value of SAA RFU values measured at 20 h, and 60 h as well as seeded αSyn oligomers as disease predictors (FIG. 4 f-i). The ROC curve demonstrates that cut-off values of 0.087 mg/mL for CSF seeded o-αSyn, 17,314 RFU for Imax at 60 h, 10,644 RFU for Imax at 20 h, and 721,500 RFU for ThT AUC are the most reliable measures to distinguish patients with PD from Ctrls. The above-mentioned cut-off values yield a sensitivity of 91.9% (95% CI, 82.4%-96.5%) and a specificity of 85.3% (95% CI, 69.8%-93.5%), with an area under the curve (ROC AUC) of 0.969 for CSF seeded o-αSyn at 20 h. However, the cut-off value for Imax at 20 h yields a sensitivity of 80.7% (95% CI, 69.1%-88.5%) and a specificity of 76.5% (95% CI, 60.0%-87.5%), with an AUC of 0.860 (Table 2).

TABLE 2
Sensitivity and specificity of α-Syn RT-QuIC assay and
seeded α-Syn oligomers in the human CSF cohort

| | PD (n = 62) | | Ctrl (n = 34) | | | | |
|---|---|---|---|---|---|---|---|
| | True (+) | False (−) | True (−) | False (+) | Sens % | Spec % | ^{a} AUC |
| ^{b} RT- | 49 | 13 | 34 | 0 | 79.0% | 100% | 98.4% |
| QuIC @ | | | | | | | |
| 60 h | | | | | | | |
| | PD (n = 62) | | Ctrl (n = 34) | | | | |
| | True (+) | False (−) | True (−) | False (+) | Sens % | Spec % | AUC |
| RT- | 20 | 42 | 34 | 0 | 32.2% | 100% | 86.0% |
| QuIC @ | | | | | | | |
| 20 h | | | | | | | |

| | PD (n = 62) | | Ctrl (n = 34) | | | | |
|---|---|---|---|---|---|---|---|
| | True (+) | False (−) | True (−) | False (+) | Sens % | Spec % | AUC |
| ^{c} ELISA @ | 58 | 4 | 27 | 7 | 93.5% | 79.4% | 96.8% |
| 20 h | | | | | | | |

Cases were scored positive solely based on exceeding the RFU threshold for RT-QuIC, or exceeding the LLOQ for ELISA.
^{a} AUC was calculated using IBM SPSS software (version 24.0; Chicago, IL).
^{b} RFU threshold for RT-QuIC at 60 h = 41479
^{c} LLoQ for ELISA = 51 pg/mL

(D) Correlation Between Disease Severity and CSF Seeded αSyn Oligomers

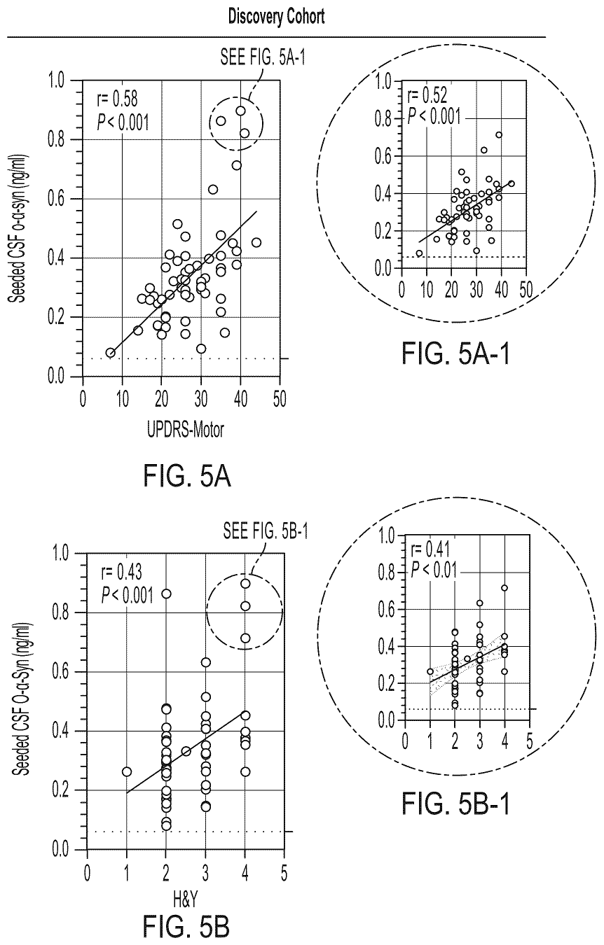

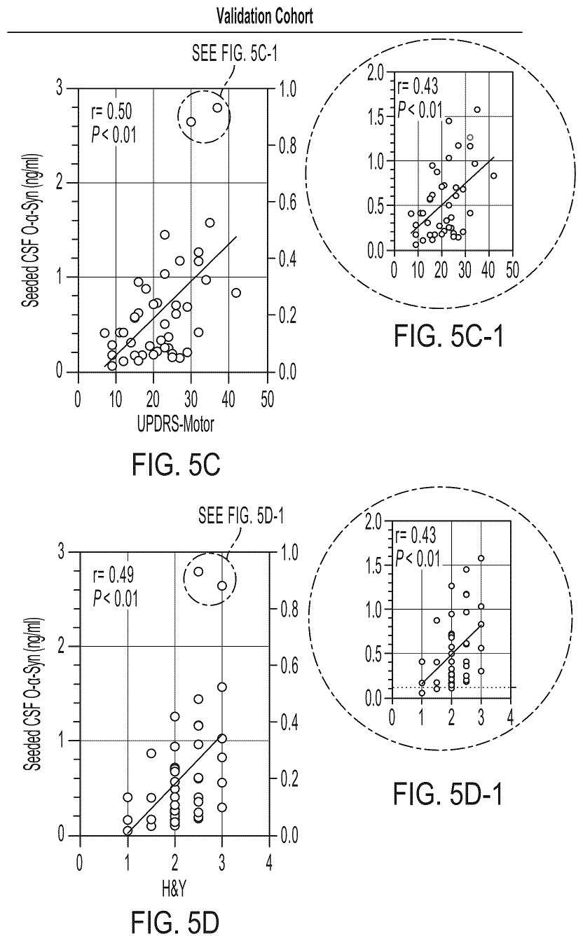

In order to investigate whether the level of αSyn oligomers in samples seeded with CSF from PD patients can reflect the severity of the disease, we explore whether SAA kinetic parameters kinetic derived from αSyn-SAA-positive PD samples correlate with CSF seeded αSyn oligomers in the discover cohort. There are no notable correlations among kinetic parameters and CSF seeded αSyn oligomers, other than a weak correlation with Imax at 20 h (r=0.25, p<0.05). Exploring the correlation between the levels of CSF αSyn oligomers at 20 h with disease severity in the PD group shows positive correlations between CSF seeded αSyn oligomers and both the unified Parkinson's disease rating scale-motor scores, (UPDRS-motor) (r=0.58, p<0.001) and the Hoehn and Yahr (H&Y) (r=0.48, p<0.001) scores (FIG. 5 a, b). To further test the strength of the correlations, the same data set is re-analysed excluding 3 data points with very high concentrations of seeded αSyn oligomers (highlighted in a circle). The correlations' strength is slightly reduced, however, still present (r=0.52, p<0.001, and r=0.41, p<0.01 with UPDRS-motor and H&Y scores, respectively). There is no correlation between CSF seeded αSyn oligomers and cognitive scores of the PD patients. Similarly, using kinetic parameters positive αSyn SAA, correlation coefficients with clinical data from PD subjects are calculated. No correlations are seen. Imax at 60 h and ThT showed weak correlation with H&Y scores, that is no longer present when the same 3 data points are excluded (FIG. 5).

As in the discovery cohort, similar correlations are calculated in the validation cohort. Positive correlations are noted between CSF seeded αSyn oligomers and UPDRS-motor (r=0.5, p<0.01), and H&Y (r=0.49, p<0.01) scores (FIG. 5 c, d). Both correlations are present even when 2 cases with very high levels of CSF seeded αSyn oligomers are excluded (r=0.50, p<0.01, and r=0.43, p<0.01 with UPDRS-motor and H&Y scores, respectively).

As described above, pilot set analysis of post-mortem BH samples shows that oligomers-specific ELISA robustly quantify SAA end product from subjects with PD or DLB with high sensitivity and specificity scores (100%). The validation set of human brain homogenates further shows that seeding activity can be detected earlier with oligomeric ELISA as the additional test readout rather than SAA alone. The combination assays provide robust information about the patients' clinical disease stage. In the discovery cohort, levels of CSF seeded αSyn oligomers correlate with the severity of the clinical symptoms of PD as measured by UPDRS-motor (r=0.58, P<0.001) and H&Y scores (r=0.43, P<0.01). Similar correlations are observed in the validation cohort between the concentrations of CSF seeded αSyn oligomers and both UPDRS-motor (r=0.50, P<0.01) and H&Y scores (r=0.49, P<0.01). At 20 h, ROC analysis yields a sensitivity of 91.9% (95% CI, 82.4%-96.5%) and a specificity of 85.3% (95% CI, 69.8%-93.5%), with an area under the curve (ROC AUC) of 0.969 for CSF seeded αSyn oligomers. A sensitivity of 80.7% (95% CI, 69.1%-88.5%) and a specificity of 76.5% (95% CI, 60.0%-87.5%), with an AUC of 0.860 is observed using ThT Imax at the same time-point. Accordingly, levels of CSF seeded αSyn oligomers, as determined by SAA and ELISA, can be useful biomarkers for diagnosis, and provide information about the disease stage by correlating with clinical measures of disease severity.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

We claim:

1. A method for monitoring Parkinson's Disease (PD) progression or Dementia with Lewy Bodies (DLB) progression comprising
(i) detecting αSyn aggregation in a sample comprising αSyn oligomers using a seed amplification assay (SAA); and
(ii) measuring the amount of αSyn in the sample of step (i) with an enzyme linked immunosorbent assay (ELISA) at a time point when the amount of αSyn oligomers in the sample of step (i) exceeds the ELISA limit of quantification.

2. The method of claim 1, wherein the sample is a cerebro spinal fluid (CSF) sample, a brain homogenate (BH) sample, a whole blood sample, a plasma sample, a serum sample, a saliva sample, or a urine sample.

3. The method of claim 1, wherein the sample is a CSF sample, which is seeded, and the progression of PD is monitored.

4. The method of claim 3, wherein the ELISA assay is conducted at a time point between about 15 hours to about 25 hours after the seeding of the sample.

5. The method of claim 3, wherein the ELISA assay is conducted at a time point about 20 hours after the seeding of the sample.

6. The method of claim 1, wherein the sample is a BH sample, which is seeded, and the progression of PD or DLB is monitored.

7. The method of claim 6, wherein the ELISA assay is conducted at a time point between about 55 hours to about 65 hours after the seeding of the sample.

8. The method of claim 6, wherein the ELISA assay is conducted at a time point about 60 hours after the seeding of the sample.

9. The method of claim 1, wherein said ELISA comprises antibodies that recognize αSyn oligomers or αSyn aggregates, and do not recognize αSyn monomers.

10. The method of claim 3, wherein levels of CSF seeded αSyn oligomers correlate with the severity of the clinical symptoms of PD as measured by the Hoehn and Yahr (H&Y) scale and the Unified Parkinson's Disease Rating Scale (UPDRS) motor scores.

11. An assay for measuring the effect of test molecules on αSyn aggregation, the assay comprising
(i) obtaining a sample from a subject;
(ii) detecting αSyn aggregation in the sample using a seed amplification assay (SAA); and
(iii) measuring the amount of αSyn in the sample of step (ii) with an enzyme linked immunosorbent assay (ELISA) at a time point when the amount of αSyn oligomers in the sample of step (ii) exceeds the ELISA limit of quantification.

12. The assay of claim 11, wherein the sample is a cerebro spinal fluid (CSF) sample, a brain homogenate (BH) sample, a whole blood sample, a plasma sample, a serum sample, a saliva sample, or a urine sample.

13. The assay of claim 11, wherein the test molecules affect αSyn aggregation directly or indirectly.

14. The assay of claim 11, wherein the test molecules that affect αSyn aggregation directly or indirectly are selected from antibodies, small molecules that interfere with αSyn aggregation, or molecules that affect pathways associated with GBA gene mutations or LRRK2 gene mutations.

15. The assay of claim 11, wherein the assay is used in pre-clinical testing.

16. The assay of claim 15, wherein the sample is a BH sample obtained from a non-human species.

17. The assay of claim 15, wherein the sample is a CSF sample, a whole blood sample, a plasma sample, a serum sample, a saliva sample, or a urine sample obtained from a non-human species.

18. The assay of claim 11, wherein the assay is used in human clinical trials.

19. The assay of claim 18, wherein the sample is a cerebro spinal fluid (CSF) sample, a whole blood sample; a plasma sample, a serum sample, a saliva sample; or a urine sample obtained from a human subject.

20. A kit for quantification of αSyn aggregates comprising
(i) a container comprising untagged or tagged monomeric full length αSyn; C-terminally truncated αSyn, N-terminally truncated αSyn, or fragments of αSyn; and
(ii) an ELISA assay that recognizes αSyn oligomers or αSyn aggregates and does not recognize αSyn monomers.
